(19) United States
(12) Patent Application Publication
(10) Pub. No.: US 2016/0228626 A1
(43) Pub. Date: Aug. 11, 2016

(54) Illumination Device for Breast Milk Pumping

(71) Applicant: Worth Innovations, LLC, Short Hills, NJ (US)

(72) Inventor: Elizabeth CASSANO, Short Hills, NJ (US)

(73) Assignee: Worth Innovations, LLC, Short Hills, NJ (US)

(21) Appl. No.: 15/016,029

(22) Filed: Feb. 4, 2016

Related U.S. Application Data
(60) Provisional application No. 62/113,041, filed on Feb. 6, 2015.
(60) Provisional application No. 62/128,692, filed on Mar. 5, 2015.

(51) Int. Cl. A61M 1/06; F21V 33/00; F21V 14/02; F21V 23/04; F21V 23/02

(52) U.S. Cl. CPC A61M 1/066; F21V 23/0464; F21V 23/02; F21V 23/04; F21V 14/02; F21V 33/0048; A61M 2205/587

(57) ABSTRACT

Apparatuses and methods for illuminating milk flow during breast milk pumping are provided. In one aspect, the apparatus is an illumination device that may comprise a light source and an attachment mechanism configured to couple the illumination device to a breast pump and/or one or more of its components in a way that allows the light source to aim into the milk flow channel. The apparatus may further comprise an activation control and a power supply. The apparatus may be configured to attach to a breast shield, a breast shield adaptor/connector, or a milk flow channel. The apparatus may be configured to be permanently or removably affixed to a breast pump and/or one or more of its components.

### CROSS-REFERENCE TO RELATED APPLICATIONS

The present application claims benefit of U.S. Provisional Application No. 62/113,041, filed Feb. 6, 2015, and U.S. Provisional Application No. 62/128,692, filed Mar. 5, 2015, all of which are herein incorporated by reference in their entireties.

### FIELD OF THE INVENTION

The invention relates generally to systems, methods and apparatuses for breast milk pumping. More specifically, the invention relates to a device which can be attached or used in conjunction with a breast pump or its accessories, which illuminates the milk flow from the user to the storage container, or bottle. The invention further relates to a breast shield incorporating a light source, which illuminates the milk flow from the user to the storage container, or bottle, when used in conjunction with a breast pump or its accessories.

### BACKGROUND

It is well established in medical literature that breast milk is the best nourishment for babies, whether they are sick, premature, or full term and healthy. The World Health Organization recommends “exclusive breast feeding for all infants for six months, and partial breastfeeding continued until two years of age.¹” Breast milk contains a combination of nutrients important for infant health and is often referred to as “liquid gold,” as every drop is valuable. This milk supports infant growth and development and has many benefits to both the mother and baby. Breast milk is free, easily digestible and contains infection-fighting white blood cells that give increased protection against infections.² In addition, children that drink breast milk have a 34 percent reduced chance of developing diabetes and a 24 percent reduced chance in becoming overweight.³ ¹ World Health Organization, Global strategy for infant and young child feeding. The optimal duration of exclusive breastfeeding, in, Geneva, World Health Organization, 2001.² Quigley M A, Kelly Y J, Sacker A. Breastfeeding and hospitalization for diarrheal and respiratory infection in the United Kingdom Millennium Cohort Study. Pediatrics 2007; 119: e837-42.³ World Health Organization, Horta, MD, PhD, Bernardo L and Cesar G. Victoria, MD, PhD Long-term Effects of Breastfeeding: A systematic review, Universidade Federal de Pelatos, Pelatos, Brazil World Health Organization, 2013.

Despite breast milk being natural, it is not often easy to provide. A baby may have difficulty latching onto the breast, and even if the baby properly latches, the baby may not receive enough milk directly from the breast if the mother is not producing enough. According to the National Center for Chronic Disease Prevention and Health Promotion Breastfeeding Report for the United States in 2013, “the percentage of US infants who begin breastfeeding is high at 77 percent . . . [however] there is concern that infants are not breastfeeding for as long as recommended . . . Of infants born in 2010, [only] 49 percent were breastfeeding at six months” and 27 percent at twelve months.⁴ Women need every tool possible to provide their babies breast milk for as long as feasible. ⁴ Centers for Disease Control and Prevention, Breastfeeding Report for the United States Atlanta, Ga., National Center for Chronic Disease Prevention and Health Promotion Division of Nutrition, Physical Activity and Obesity, 2013.

While not impossible, it is difficult to breastfeed a baby for an extended period of time without using a breast pump and is highly impractical for women working outside the home. Breast pumps allow a user to pump, or express milk and later feed the baby the expressed milk. Some mothers may prefer feeding with a bottle or want to allow someone else to feed the baby. Feeding a baby pumped milk allows the baby to still receive the nutrients of the breast milk with the additional knowledge of how much milk the baby is drinking. In addition, many mothers return back to work after maternity leave and want to continue providing breast milk to their babies and using a breast pump allows working mothers this option. If a nursing woman repeatedly leaves milk in her breasts without draining them, her body will produce less and less milk. Consequently many women working outside the home must attempt breast pumping three to four times during a typical workday to maintain their milk production.

When a breast pump user is attempting to pump milk, it is an all too common occurrence that the user cannot adequately view the milk flow from the breast through the breast pump accessories into the storage container. The lighting may not be sufficient surrounding the pumping area, the breast pump pieces and accessories may be too opaque to view the milk flow and/or the user may not realize how effectively the pump is functioning. When properly running, breast pumps make noticeable, repeated noises and also place strain on the breasts. In general, larger motors produce less noise, and smaller motors are louder. During the pumping process, a woman may feel the initial “let down” of her milk, however the last few minutes of pumping often are indistinguishable between the milk flowing and the breast pump pumping “dry.” The prior art suggests that the user should attempt to pump milk for a set period of time or until the user thinks the milk is no longer flowing.

This lack of understanding of the actual milk flow may result in the user not fully draining the breasts and consequently not providing the full supply of milk to the baby. Breast milk increases in fat content throughout the expressing process and the later breast milk is higher in fat content; milk that is inadvertently left in the breast is the most nourishing and valuable to the baby. Every last drop of breast milk provides needed nourishment. When milk is repeatedly left in the breast, from either not breastfeeding or from not pumping, the body produces less milk as the body adapts to the level of milk expressed.

Determining the milk flow can be difficult. The volume of milk available to pump from the breasts varies based on the time of day, duration between pumping, the capabilities of the mother and various additional factors. A breast pump user may have a general understanding of how long it takes to express milk, but without viewing the actual milk flow, the user will either lose valuable time by continuing to attempt to pump breasts that have already fully been drained, or will unknowingly stop pumping and leave milk in the breasts, depriving the baby of the remaining milk and its nutrients.

Accordingly, there exists a need for a method and apparatus to illuminate the milk flow from a breast pump and the associated accessories.

### SUMMARY

The present disclosure describes an illumination device for lighting the expressed milk flow from a breast pump. An apparatus according to the present disclosure comprises, without limitation, an attachable/detachable lighting source with an activation mechanism and power supply that allows the user to view the milk flow more clearly and adjust pumping accordingly providing a better understanding of the continuity of the milk flow. Another apparatus according to the present disclosure, comprises without limitation, a breast shield with a lighting source incorporated into the breast shield and/or connector with an activation mechanism and power supply that allows the user to view the milk flow more clearly and adjust pumping accordingly providing a better understanding of the continuity of the milk flow. A method of expressing milk using a breast pump attached to an illumination device is also provided. Another method of expressing milk using a breast pump attached to an illuminating breast shield/flange and/or connector device is also provided.

### BRIEF DESCRIPTION OF THE DRAWINGS

These and other features and advantages of the present disclosure will become more readily appreciated and better understood by reference to the following detailed description when considered in connection with the accompanying drawings which are not to scale, wherein:

FIG. 1 is a user pumping milk with an exemplary breast pump assembly, including breast shield, breast shield connector/adaptor, tube and collection container attached to an exemplary embodiment of an illumination device in accordance with the present disclosure for illuminating the milk flow;

FIG. 2 is a more detailed view of the components of an exemplary breast shield and a breast shield connector/adaptor with tube, which is attached to an exemplary embodiment of an illumination device with a light source, attachment mechanism, device activation control, light source adjustment control, optically coated reflector, power supply and communications transceiver in accordance with the present disclosure;

FIG. 3 is an interior view of an exemplary illumination device as depicted in FIG. 2 in accordance with the present disclosure;

FIG. 4 is another view of an exemplary illumination device in accordance with the present disclosure as depicted in FIG. 2 communicating with an external communications device.

FIG. 5 is a view of an embodiment of an illumination device, whereby the illumination device is directly incorporated into the milk flow channel.

### DETAILED DESCRIPTION

Referring to the Figures, wherein like numerals indicate like or corresponding parts throughout the several views. For purposes of illustration and not to be in any way limiting, the following description will make reference to electric breast pumps, however it will be appreciated that the invention is equally applicable to manual breast pumps.

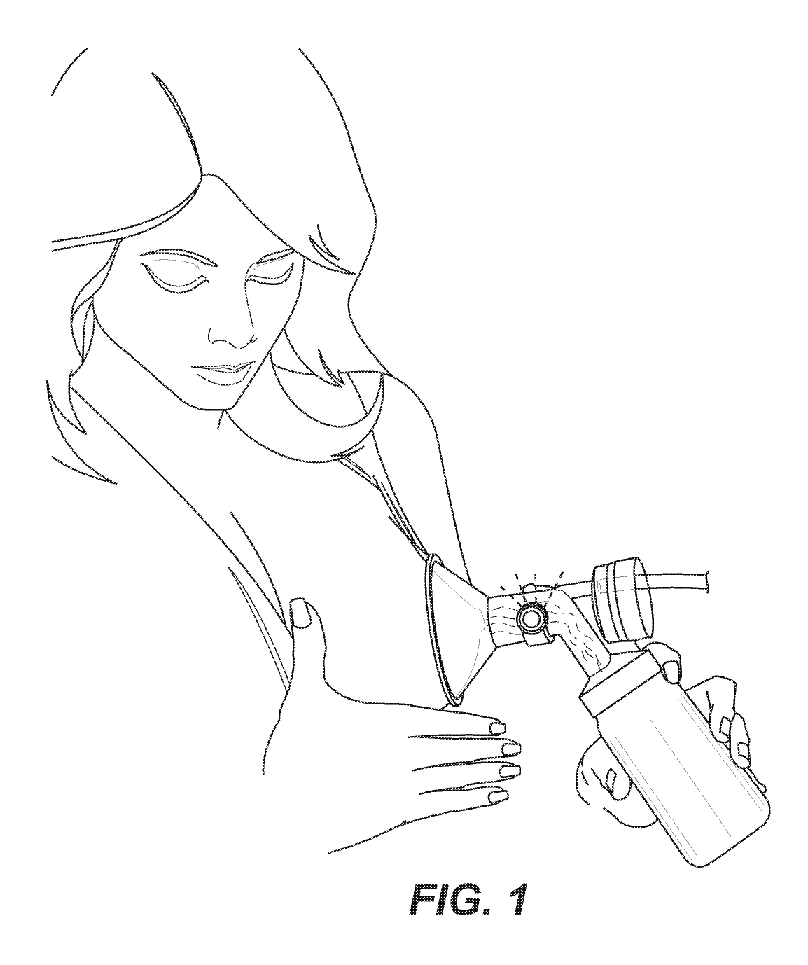

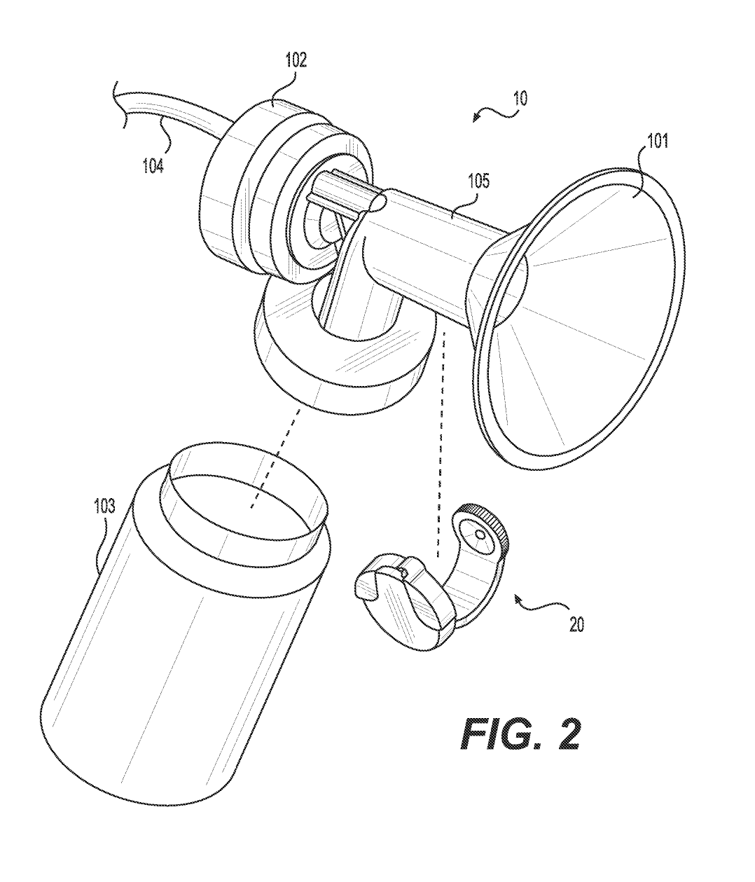

An exemplary breast pump assembly, as known in the art, is depicted as part 10 in FIGS. 1 and 2. It will be understood that this is one example of a breast pump assembly, but that there are other variations on breast pump assemblies known in the art. A typical breast pump assembly 10 may comprise a breast shield 101, a breast shield connector/adaptor 102, and a collection container 103 for collecting milk. A typical breast shield connector/adaptor 102 has a hole in the end for the tube 104 which connects to the pump and power supply as shown in FIG. 1 and FIG. 2. In some embodiments, a breast shield 101 and/or breast shield connector/adaptor 102 may comprise a milk flow channel 105.

As used herein, a milk flow channel refers to the “tunnel” between the user's breast while cupped by the breast shield, and the collection container that the milk travels through. The milk flow channel may comprise portions of the breast shield and the breast shield connector/adaptor.

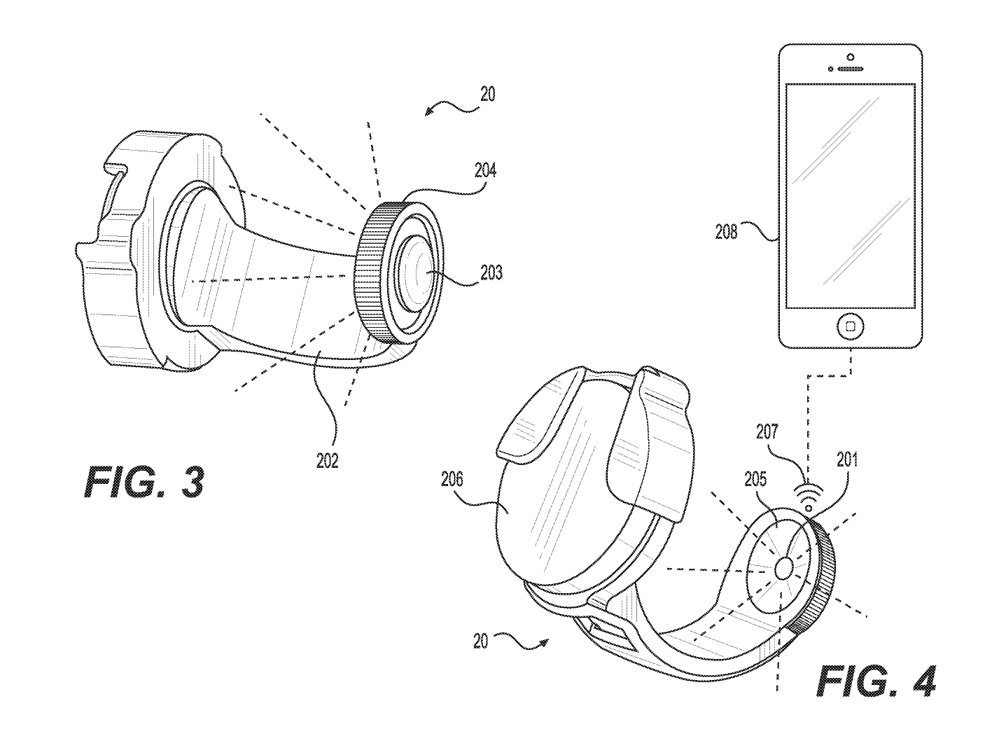

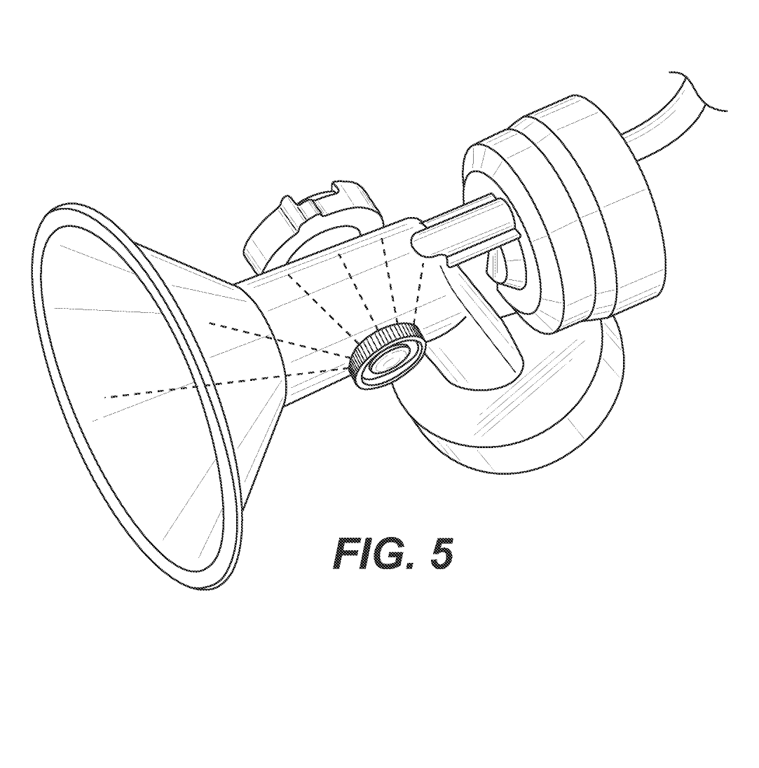

An exemplary illumination device according to the present disclosure is shown in FIGS. 2 through 5 as part 20. By way of non-limiting example, an illumination device 20 according to the present disclosure may comprise a light source 201, an attachment mechanism 202, a device activation control 203, a light source adjustment control 204, an optically coated reflector 205 and a power supply 206. In certain embodiments according to the present disclosure, the suitable light source 201 would be from one or more light-emitting diodes, halogen, xenon, and/or incandescent light bulbs all emitting, by way of example 15-50 lm/W. In some embodiments, the suitable light source 201 has a luminous efficacy of 15-20 lm/W, 20-25 lm/W, 25-30 lm/W, 30-35 lm/W, 35-40 lm/W, 40-45 lm/W, or 45-50 lm/W. In another embodiment, the suitable light source 201 has a luminous efficiency of 15-45 lm/W, 15-40 lm/W, 15-35 lm/W, 15-30 lm/W, or 15-25 lm/W. In yet another embodiment, the suitable light source 201 has a luminous efficiency of 20-50 lm/W, 25-50 lm/W, 30-50 lm/W, 35-50 lm/W, or 40-50 lm/W. In certain embodiments, the suitable light source 201 may emit ultraviolet light, visible light, infrared, or a combination thereof. In some embodiments, the suitable light source 201 may emit visible light having wavelengths ranging from 380 to 800 nm. In another embodiment, the suitable light source 201 emits light having wavelengths ranging from 380 nm to 450 nm, from 450 nm to 495 nm, from 495 nm to 570 nm, from 570 nm to 590 nm, from 590 nm to 620 nm, or from 620 nm to 750 nm. In a further example, another embodiment of this light source 201 would be powered from solar energy by way of an appropriately sized solar panel based on the size of the illumination device 20. The light sources 201 described are by way of non-limiting example and are not limited by the available technology of the day as far as sources of possible light 201.

In certain embodiments according to the present disclosure, the illumination device 20 may comprise an attachment mechanism 202, by way of which the illumination device 20 may attach to the breast pump assembly 10 and/or its components in a manner suitable to permit enhanced illumination of the breast milk and/or milk flow. In another embodiment, the illumination device 20 may comprise an attachment mechanism 202, by way of which the illumination device 20 may attach to a breast shield 101 in a manner suitable to permit enhanced illumination of the breast milk and/or milk flow. By way of non-limiting example, in one embodiment the attachment mechanism 202 may couple directly to the breast shield connector/adaptor 102. In yet another embodiment, the attachment mechanism 202 may couple to the collection container 103. In still yet another embodiment, the attachment mechanism 202 may couple directly to the breast pump, and the like.

In certain embodiments, the attachment mechanism 202 may removably couple the illumination device 20 to the breast pump. In such embodiments the attachment mechanism 202 may be anything suitable for removably coupling the illumination device 20 to a breast pump including, without limitation, a claw-type snapping mechanism, friction, a temporary, reusable adhesive or other fastener, one or more magnets, a twisting mechanism or a compression mechanism. For illustration purposes, a non-limiting example of a compression attachment mechanism 202 includes, but is not limited to, an adjustable edge surrounding the rim of the illumination device 20, such as a rubber edge. When a compression is made on another segment of the device 20, the diameter of the outer edge would decrease such that the outer edge can thread into the breast pump opening. For purposes of illustration, a non-limiting example of the illumination device 20 with a twisting attachment mechanism 202, has a threaded ring around the outer edge which threads into the corresponding threads on the breast shield connector/adaptor 102. In such embodiments, the attachment mechanism 202 may comprise, but are not limited to, snapping, magnetic, screwing, friction, twisting, compressing use of an adhesive, or other fastener.

In certain embodiments, the attachment mechanism 202 may removably couple the illumination device 20 to the breast shield 101. For purposes of illustration, a non-limiting example of the illumination device 20 with a compression attachment mechanism 202 couples around the milk flow channel 105. In such embodiments the attachment mechanism 202 may be anything suitable for removably coupling the illumination device 20 to the breast shield 101 including, without limitation, a claw-type snapping mechanism, friction, a temporary, reusable adhesive or other fastener, one or more magnets, a twisting mechanism or a compression mechanism 202. For purposes of illustration, a non-limiting example of the illumination device 20 with a twisting attachment mechanism 202, has a threaded ring around the outer edge which threads into the corresponding threads on the breast shield connector/adaptor 102. A non-limiting example of a compression mechanism includes, but is not limited to, an adjustable edge surrounding the rim of the illumination device 20, such as a rubber edge. When a compression is made on another segment of the device 20, the diameter of the outer edge would decrease such that the outer edge can thread into the breast shield 101 opening. In such embodiments, the attachment mechanism 202 may comprise, but are not limited to, snapping, magnetic, screwing, friction, twisting, compressing use of an adhesive, or other fastener.

In other embodiments according to the present disclosure, the illumination device 20 may comprise an attachment mechanism 202 that is permanently affixed on one or more of the components of the breast pump in a manner suitable to permit enhanced illumination of the breast and/or milk flow.

The illumination device 20 may further comprise an activation control 203, to turn on and off the light source 201 during breast milk pumping. In certain embodiments, this activation control 203 may automatically determine when to turn the light source 201 on or off using, for example, a sensor which detects the milk flow. Without limitation, the sensor could be controlled by the user, pump or an electronic device such as a phone or a wearable device. A non-limiting example of a sensor trigger is the indication of milk moving out of the breasts. In some embodiments, the sensor may be an optical sensor or a mechanical sensor. In certain embodiments, an optical sensor may be a ambient light sensor, a ultraviolet light sensor, or an infrared sensor. In further embodiments, a mechanical sensor may be a flow sensor. In other embodiments, the illumination device 20 may further comprise a manual on/off mechanism 203, such as a switch or push button.

In certain embodiments, according to the present disclosure, the light source 201 has an adjustable control 204. This adjustment comprises, but is not limited to, adjusting for varying levels of brightness and/or the directionality of the light source 201. The brightness of the light source 201 increases or decreases manually and/or automatically based on the available light surrounding the user. In some embodiments, the available light surrounding the user is determined by a luminosity sensor mounted on the illumination device, breast pump or breast pump accessories. By way of non-limiting example, the exemplar directionality of the light source 201 would be angled straight onto the nipple portion of the breast. In another embodiment the angle of the light source 201 would adjust manually through the light source adjustment control 204 by the user to illuminate the milk flow. In yet another embodiment, the light source 201 would adjust automatically through the adjustment control 204, sensing the directionality and volume of the milk flow. In another embodiment, the light source 201 would indirectly illuminate the milk flow.

In certain embodiments, the light source 201 of the illumination device 20 in the present disclosure has an optically coated reflector 205 behind it to enhance the illumination.

An illumination device according to the present disclosure may further comprise a power supply 206 suitable to power the light source 201. In certain embodiments, this may be a self-contained power supply 206, such as a battery, to allow hands-free usage of the illumination device 20. In other embodiments, the power supply 206 may couple with the power supply 206 of the breast pump, or it may couple to an external power supply 206 including the sun.

In certain embodiments, an illumination device 20 according to the present disclosure may further comprise a communications transceiver 207, such as, but not limited to, a Bluetooth or Wifi transceiver, to permit communications with a phone or other external communications device 208 including but not limited to a computer, tablet or other mobile or wearable device, during the breast pumping process. In some embodiments, the communications transceiver 207 is capable of transmitting signals from a sensor to an external communications device 208. In certain embodiments, the external communications device 208 is capable of displaying data about speed of milk expression and quantity of expressed milk. In another embodiment, the communications transceiver 207 is capable of receiving signals from an external communications device 208 to control the illumination device 20.

By way of illustration, not limitation, the illumination device 20 outer components would be fashioned of light-weight, durable, waterproof, Bisphenol A-free plastic and/or metals, by way of non-limiting examples, aluminum, or titanium, such that as the illumination device 20 is in use, the outer parts remain cool to the touch. The approximate weight of the illumination device 20, according to the present disclosure would weigh between 0.5 and 2 ounces. In some embodiments, the illumination device 20 may weigh between 0.5 ounce to 1 ounce, 1 ounce to 1.5 ounces, or 1.5 ounces to 2 ounces. By way of non-limiting example, the approximate size of the illumination device 20 would be 1 to 2 inches in diameter and 1.5 to 3 inches long. In some embodiments, the illumination device 20 may have a diameter ranging from 1 inches to 1.5 inches, or from 1.5 inches to 2 inches. In another embodiment, the illumination device 20 may have a length ranging from 1.5 inches to 2.0 inches, from 2.0 inches to 2.5 inches, or from 2.5 inches to 3.0 inches.

By way of non-limiting illustration of an example embodiment of the device described herein, the user would utilize the illumination device 20 in the following manner. Attach the illumination device 20 onto the milk flow channel 105 through the compression attachment mechanism 202 in the location indicated in FIG. 2 with the light source 201 aimed into the milk flow channel 105. Utilizing the device activation control 203, the user can turn on the power supply 206 to the illumination device 20. The light source 201 will activate and the optically coated reflector 205 magnifies the light. The user may adjust the directionality or intensity of the light source 201 as necessary with the light source adjustment control 204. The user can attach the tube 104 from the breast pump into the breast shield connector/adaptor 102 as known in the art. The user may then activate the power supply of the breast pump. As the milk is expressed, the user views the milk flow through the milk flow channel 105, into the breast shield connector/adaptor 102, and into the attached collection container 103. As the milk is flowing, the communications transceiver 207 in the illumination device 20 tracks the volume of milk and time elapsed while pumping and sends it to the user's external device 208. As the milk is expressed, the user can massage the breast to maximize the milk flow through the milk ducts. By utilizing the illumination device 20, the user visually determines that the milk is no longer flowing. Then the user turns the breast pump off and deactivates the illumination device 20 with the activation control 203. The user then separates the breast shield 101, breast shield connector/adaptor 102, collection container 103 and tube 104 from the breast. The tube 104 is removed from the breast shield connector/adaptor 102 and finally the illumination device 20 is separated from the breast shield connector/adaptor 102. The user then reviews the collected pumping data from the external device 208.

It will be understood that the use of the apparatus described herein is not limited to determining the milk flow, but can be used for other purposes during milk expressing with a breast pump that the user deems appropriate.

The device has been described in an illustrative manner, and it is to be understood that the terminology which has been used is intended to be of the nature of words of description rather than of limitation. Modifications and variations of the present invention are possible in light of the above teachings. It is, therefore, to be understood that within the scope of the appended claim, the invention may be practiced otherwise than as specifically described. The invention is defined by the claims.

What is claimed is:

1. An illumination device for viewing milk flow during breast milk pumping comprising:
a light source emitting visible light;
an attachment mechanism configured to couple the illumination device to a breast pump and/or one or more of its components in a way that allows the light source to aim into the milk flow channel;
an activation control configured to turn on and off the light source; and
a power supply.

2. The illumination device of claim 1, wherein the attachment mechanism is configured to couple the illumination device to a breast pump component selected from the group consisting of a breast shield and a breast shield adaptor.

3. The illumination device of claim 2, wherein the breast pump component is a breast shield adaptor, and the light source is configured to aim into the milk flow channel through a breast shield connector.

4. The illuminated breast shield of claim 2, wherein the breast pump component is a breast shield, and the light source further comprises an optically coated reflector at the rear to enhance the illumination.

5. The illumination device of claim 1, wherein the breast pump and/or one or more of its components is a milk flow channel, and wherein the illumination device is permanently affixed to the milk flow channel.

6. The illumination device of claim 1, wherein the breast pump and/or one or more of its components is a milk flow channel, and wherein the illumination device is removably affixed to the milk flow channel.

7. The illumination device of claim 1, wherein the light source brightness is manually adjustable.

8. The illumination device of claim 1, further comprising a luminosity sensor, and wherein the light source brightness is configured to adjust automatically based on the available light detected by the luminosity sensor.

9. The illumination device of claim 1, wherein the light source directionality is manually adjustable.

10. The illumination device of claim 1, further comprising a milk flow sensor, and wherein the light source directionality is configured to adjust automatically based on the directionality of milk flow detected by the milk flow sensor.

11. The illumination device of claim 10, wherein the milk flow sensor is selected from the group consisting of an optical sensor and a mechanical sensor.

12. The illumination device of claim 1, wherein the activation control utilizes a sensor that detects milk flow.

13. The illumination device of claim 1, further comprising a communications transceiver.

14. The illumination device of claim 13, wherein the communications transceiver is configured to receive signals from an external device to control the illumination device.

15. The illumination device of claim 13, further comprising a sensor selected from the group consisting of a luminosity sensor and a milk flow sensor, and wherein the communications transceiver is configured to transmit signals from the sensor to the external device.

16. The illumination device of claim 15, wherein said external device is configured to display the speed of milk expression and quantity of expressed milk based on the transmitted signals.

17. The illumination device of claim 1, wherein the power supply is selected from the group consisting of a self-contained power supply and a power supply from the breast pump.

18. A method of expressing milk comprising:
providing an illumination device comprising:
a light source emitting visible light; and
an attachment mechanism configured to couple the illumination device to a breast pump and/or one or more of its components in a way that allows the light source to aim into the milk flow channel;
expressing milk using a breast pump that attaches to the breast shield;
viewing the milk expression through the breast shield; and
turning off the breast pump when milk is no longer flowing.

19. The method of claim 18, wherein the breast pump component is a breast shield adaptor, wherein the illumination device is permanently affixed to the breast shield adaptor, and wherein the light source is configured to aim into the milk flow channel through a breast shield connector.

20. The method of claim 18, wherein the breast pump component is a breast shield, wherein the illumination device is permanently affixed to the breast shield, and wherein the light source further comprises an optically coated reflector at the rear to enhance the illumination.
